(19) United States
(12) Patent Application Publication
(10) Pub. No.: US 2016/0216192 A1
(43) Pub. Date: Jul. 28, 2016

(54) CELL CULTURING AND TRACKING WITH OLED ARRAYS

(71) Applicant: EMPIRE TECHNOLOGY DEVELOPMENT LLC, Wilmington, DE (US)

(72) Inventor: Yansong Gu, Bellevue, WA (US)

(73) Assignee: Empire Technology Development LLC, Wilmington, DE (US)

(21) Appl. No.: 14/916,743

(22) PCT Filed: Sep. 5, 2013
(86) PCT No.: PCT/US2013/058244

§ 371 Date: Mar. 4, 2016

(51) Int. Cl. G01N 15/02; G01N 33/50; G01N 21/64; H05B 33/08; G01N 15/14

(52) U.S. Cl. CPC G01N 15/0205; H05B 33/0896; G01N 15/1434; G01N 21/6486; G01N 33/5008; G01N 2015/1493; G01N 2201/0628

(57) ABSTRACT

Cell culturing and tracking systems using an array of organic light emitting diodes (OLEDs) to illuminate cells and/or other particles in a cell chamber are described. Compared to conventional light sources, the OLED array consumes very little energy and emits a small amount of waste heat, so it may be disposed near or on the cell chamber. For instance, it can be printed on one side of the cell chamber itself. In addition, the OLED array may be patterned into pixels or sub-pixels (individual OLEDs), each of which is as small as or smaller than an individual cell or particle. Because the pixels are so small, OLED illumination can be used to acquire images with a spatial resolution equal to or better than the cell or particle cell. As a result, the OLED array can be used to track, monitor, identify, and manipulate individual cells within the cell culture.

### BACKGROUND

Flow cytometry is used in research and clinical diagnosis to sort biological cells and other particles. In flow cytometry, a monochromatic beam of light illuminates part of a liquid stream that includes one or more particles. As a particle in the liquid stream passes through the illumination region, it scatters light and/or fluoresces towards one or more detectors, which sense variations in the amplitude and wavelength of the scattered light and the fluorescent light. These variations can be used to determine the particle's size, position, and composition.

### SUMMARY

Despite many improvements and upgrades over the years, flow cytometry technology has certain limitations and shortcomings. For example, flow cytometry works well for sorting mixed cell populations in suspension, such as blood cells. It does not work for individual cells or populations of a few cells, such as transitional cells during stem cell differentiation, post mitotic neuronal cells, and hepatocytes in primary culture. In those cases, researchers have to employ fluorescent labeling and imaging techniques to visualize and characterize individual cells. By then, the cells are fixed and dead.

Embodiments of the present technology address limitations and shortcomings of conventional flow cytometry and cell culturing. For instance, one embodiment comprises a system for illuminating at least one cell and a corresponding method of illuminating at least one cell. In one example, the system comprises a transparent substrate having a thickness of about 10 nm to about 100 μm, an array of organic light-emitting diodes (OLEDs), and a controller. In operation, the OLED array illuminates the cell with light via the transparent substrate, and the controller, which is operably coupled to the array of OLEDs, controls the intensity, the wavelength, or both of the light emitted by the array of OLEDs.

Another embodiment comprises a system for culturing and/or tracking at least one cell. An example of this system includes a transparent substrate, a two-dimensional array of OLEDs, an active matrix layer electrically coupled to the OLED array, a detector, and a processor operably coupled to the active matrix layer and the detector. The transparent substrate defines a first surface to at least partially support the cell and a second surface, opposite the first surface, upon which the two-dimensional array of OLEDs is disposed. In operation, the two-dimensional array of OLEDs is actuated by the active matrix layer and used to illuminate the cell. The detector senses light that is transmitted, reflected, scattered, and/or emitted by the cell, and the processor controls the array of OLEDs based at least in part on the light sensed by the detector.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

The accompanying drawings, which are incorporated in and constitute a part of this specification, illustrate embodiments of the disclosed technology and together with the description serve to explain principles of the disclosed technology.

FIG. 1 shows a system for culturing and tracking individual live cells using organic light-emitting diodes (OLEDs).

FIG. 2 is an exploded view of a chamber slide printed with an active-matrix OLED array suitable for use with cell culturing and tracking system of FIG. 1.

FIG. 3 is a photograph of an active-matrix OLED array with different pixels switched on and off to form the letters “g” and “u.”

### DETAILED DESCRIPTION

The present disclosure describes an innovative platform technology that addresses the efficacy of high throughput screening (HTS), high-content screening (HCS), and high-content analysis (HCA) and may be used to reveal unknown cell behaviors. Embodiments of this platform technology may be used in basic research as well as in the pharmaceutical, biotech, and clinical diagnosis industries. More specifically, examples of the cell culturing and tracking apparatus disclosed herein may be used to discover and research different cell behaviors, many of which are difficult to predict. An exemplary cell culturing and tracking apparatus may also be used to increase the sensitivity of HTS thanks to spatial resolution on the order of the size of an individual cell. In clinical diagnosis, an exemplary cell culturing and tracking apparatus can be used to identify individual residual cancer cells or other rare cells.

Unlike conventional flow cytometry and cell sorting techniques, which rely on statistical analysis of cell populations, an exemplary cell culturing and tracking system can analyze and activate a single cell at a time. This ability to resolve a single cell may allow new discoveries of the behavior of cells in response to controlled light. For example, one may activate a single somatic cell and reprogram it into a stem cell, and/or activate a stem cell for directed differentiation.

Systems for Cell Culturing and Tracking

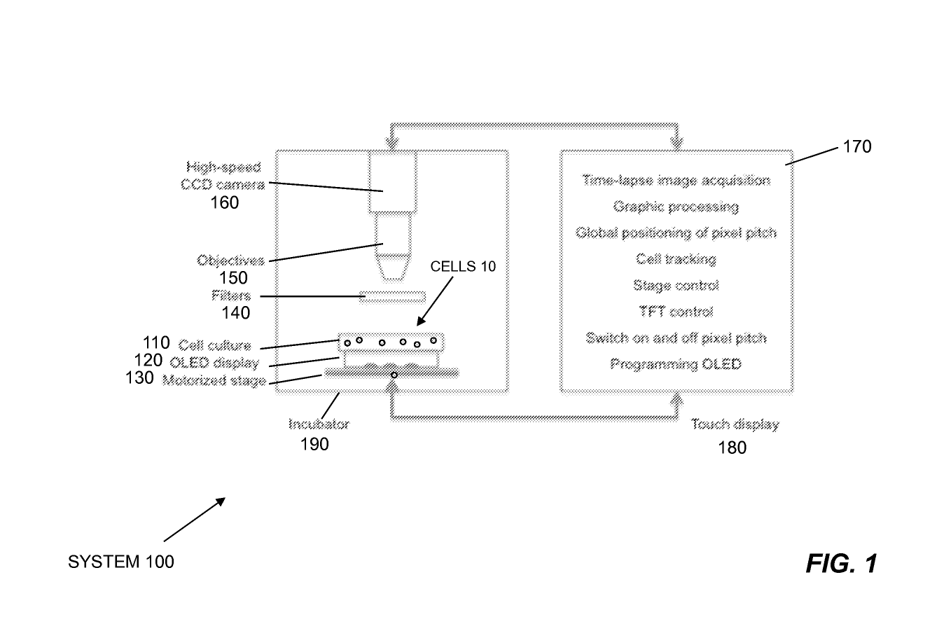

FIG. 1 shows a system 100 for culturing and tracking live cells and other particles. The system 100 includes a transparent or translucent cell culture dish 110, an organic light-emitting diode (OLED) display 120, and a motorized translation/rotation stage 130 in an incubator 190. The incubator 190 also contains one or more filters 140, one or more objective lenses 150, and a high-speed detector array 160, such as a charge-coupled device (CCD) array or a complementary metal-oxide-semiconductor (CMOS) array. A processing module 170 and a touch display 180 are coupled to components inside the incubator 190 via suitable connections (for example, cables or wireless connections).

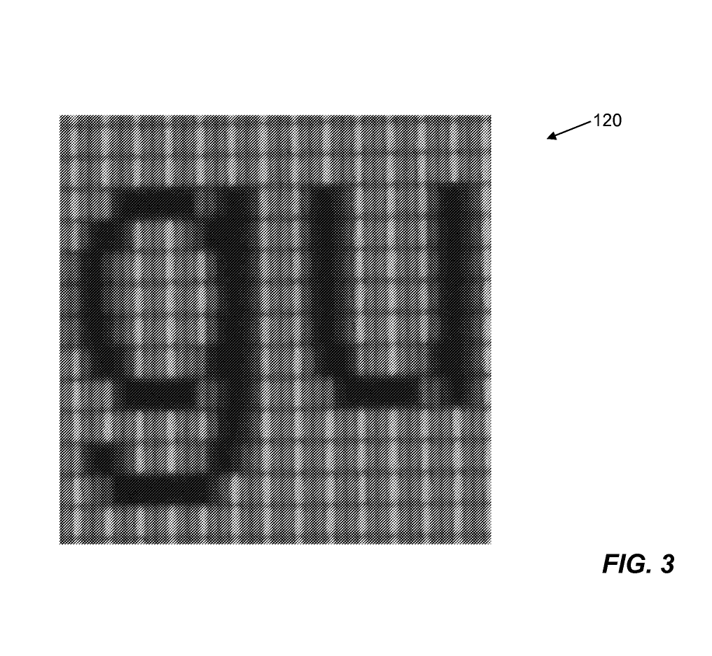

In operation, the cell culture dish 110 holds one or more cells 10, which may form a cell culture. The cells 10 may adhere to the walls of the cell culture dish 110 and/or move within fluid also disposed in the cell culture dish 110. In some cases, the cell culture dish's interior surfaces may be textured or treated to promote adhesion of the cells and/or cell growth. When a stem cell line is seeded in the cell culture dish 110, stem cells may proliferate as colonies in three dimensions and/or differentiate and migrate in two dimensions. When illuminated with light from a monochromatic OLED array (for example, OLED array 120), the cells may block the light intensity and total flux, rendering the landscape of stem cells visible. When a separate active matrix OLED (AMOLED) unit (not shown) is attached to the cell culture dish 110, the individual subpixels in OLED array 120 can be switched on and off, for example, as shown in FIG. 3. In this way, individual stem cells or differentiating and migrating cells will be captured and monitored under a microscope (lens 150 and detector array 160). If the stem cells are labeled with one or more fluorescent probes, they may emit fluorescent light that can be detected with the detector array 160.

The cell culture dish 110 sits on an organic light-emitting diode (OLED) display 120, which may be separate from or integrated into or onto the cell culture dish 110. Unlike incandescent bulbs and arc lights, the OLED array 120 consumes little energy and dissipates very little heat, so it is less likely to damage the cells 10, even when disposed in close proximity to or directly on the cell culture dish 110. For instance, the OLED array 120 may be printed onto one or more of the cell culture dish's exterior surfaces, including a curved exterior surface. In addition, by using an OLED array 120 instead of a conventional light source, it becomes possible to illuminate the cell culture from within the incubator 190 without disrupting the temperature, moisture, and gases of the cell culture environment. In this way, researchers can easily acquire reliable live cell images.

As understood by those of skill in the art, an OLED is a light-emitting diode (LED) in which the emissive electroluminescent layer is a film of organic compound that emits light in response to an electric current. Active-matrix OLEDs (AMOLED) use a thin-film transistor (TFT) backplane to switch each individual pixel on or off, but allow for higher resolution and larger display sizes. Unlike a liquid crystal display (LDC), the OLED 120 array does not require a backlight. Thus, it can display deep black levels and can be thinner and lighter than an LCD. In low ambient light conditions, such as a dark room, the OLED display 120 screen can achieve a higher contrast ratio—because it does not use a backlight, black is truly the absence of light—than an LCD, whether the LCD uses cold cathode fluorescent lamps or an LED backlight. The OLED display 120 also has a relatively low thermal conductivity, so it typically emits less light per area than an inorganic LED display.

As described in greater detail below, the OLED array 120 may include a plurality of OLEDs (pixels), each of which emits light through the cell culture dish 110 and towards the cells 10. As understood by those of skill in the art, an OLED is a light-emitting diode (LED) in which the emissive electroluminescent layer comprises a film of an organic compound that emits light in response to application of an electric current. In some embodiments, each pixel in the OLED array 120 may be about the same size as one of the cells 10 in the cell culture dish 110, for example, about 1 μm², 2 μm², 5 μm², 10 μm², 25 μm², 50 μm², or 100 μm². The pixels in the OLED array 120 may be arranged in a rectilinear area, circular array, sparse array, or any other type of periodic or aperiodic array. For instance, the OLED array 120 may comprise an array with 10 pixels×10 pixels, 100 pixels×100 pixels, 1000 pixels×1000 pixels, or any other suitable number of pixels. Each pixel may be actuated independently using active-matrix addressing or any other suitable control scheme as explained below.

When illuminated, the cells 10 transmit, scatter, and/or absorb at least some of the light emitted by the OLED array 120. The exact degrees of transmission, scattering, and absorption may vary and can depend on the cell's size, internal structure, composition (refractive index), and orientation with respect to the pixels in the OLED array 120 that are emitting light. For example, spherical cells or particles may scatter light at different angles than oblong particles or cells.

One or more of the cells 10 may also fluoresce at a first wavelength in response to illumination at a second wavelength. For instance, illuminating cells 10 expressing green fluorescent protein (GFP) with blue or ultraviolet light yields emission at a wavelength of about 509 nm. Cells 10 that do not express GFP may not fluoresce at the same wavelength in fact, they may fluoresce at other wavelengths (for example, in the red portion of the visible spectrum) if at all.

As shown in FIG. 1, the high-speed camera 160 senses light transmitted, scattered, and/or emitted by at least some of the illuminated cells 10 in the cell culture via a filter 140 and an objective lens 150. Both the filter 140 and the objective lens 150 may be moved into and out of the optical path via a respective wheel (not shown). In some cases, the system 100 may include several objective lenses 150 (for example, 0.5× to 40× conventional microscope objectives) held by a revolving nose installed in the incubator 190. Likewise, the system 100 may include a revolving filter wheel (not shown) that holds several filters 140, each of which has a different neutral density (attenuation value) and/or transmission wavelength (for example, 509 nm). If desired, the filter 140 may block light emitted by the OLED array 120 at the excitation wavelength (for example, it may block blue or ultraviolet light) and transmit fluorescent light (for example, green light) emitted by one or more of the cells 10 to prevent the excitation light from saturating the camera 160.

In operation, the camera 160 acquires one or more images of at least some of the illuminated cells 10. Depending on which pixels in the OLED array 120 are active, the location and distribution of the cells 10, the size(s) of individual cells 10, and the image resolution, the camera 160 may be able to resolve one or more individual cells 10 at a time. The camera 160 transmits the image data to the processing module 170, which collects and processes time-lapse images of the cells 10.

In some examples, the processing module 170 processes the image data from the camera 160 to estimate the cells' positions, trajectories, and/or velocities. For instance, the processing module 170 may estimate a given cell's size and center based on the number and centroid, respectively, of illuminated pixels in the image. It may estimate the cell's trajectory and velocity based on changes in the centroid's location from image to image. Additionally, it can determine fluorescence amplitude and spectrum based on information about the wavelength(s) of light emitted by the OLED array 120 and the image data. If desired, the processing module 170 may use the estimates of position, trajectory, velocity, fluorescence amplitude, and fluorescence wavelength to identify one or more of the cells 10 or particles in the cell culture dish 10.

The processing module 170 may also actuate the OLED array 120 according to a program stored in its (nonvolatile) memory, user commands received via the touch display 180 or any other suitable interface, and/or in response to the processed image data. For instance, the processing module 170 may turn certain pixels in the OLED array 120 on or off to facilitate tracking and/or identification of a particular cell 10 or particle. The processing module 170 may also cause a particular pixel in the OLED array 120 to emit more or less light, to emit light at one or more different wavelengths, and/or to blink in a particular sequence. The processing module 170 may use this functionality to create temporally and/or spatially varying illumination patterns to facilitate identification and tracking of cells 10 and particles in the cell culture dish 110. Thus, the cell culturing and tracking system 100 can be used for identification, characterization, cell counting, manipulation, and programming of individual live cells in real time.

The processing module 170 is also operably coupled to the motorized stage 130, which controls the position of the cell culture dish 110 with respect to the camera 160. The motorized stage 130 can translate the cell culture dish 110 laterally and vertically and rotate it about its vertical and lateral axes. If desired (for example, in response to user commands, pre-programmed instructions, and/or processed image data), the processing module 170 may command the motorized stage 130 to shift, and/or rotate, and/or move up or down the cell culture dish 110 with respect to the camera 160, for example, to bring a particular object or region into focus or to track a moving cell 10 or particle.

As readily understood by those of skill in the art, the processing module 170 may comprise one or more processors, including but not limited to central processing units (CPUs), graphics processing units (GPUs), microprocessors, application-specific integrated circuits (ASICs), or field-programmable gate arrays (FPGAs) as well as any appropriate bus or routing hardware. The processing module 170 may also include a volatile memory and/or a nonvolatile memory.

Further, the processing module 170 may be embodied in any of a number of forms, such as a rack-mounted computer, a desktop computer, a laptop computer, or a tablet computer. Additionally, the processing module 170 may be embedded in or embodied by a device suitable processing capabilities, including a Personal Digital Assistant (PDA), a smart phone or any other suitable portable or fixed electronic device.

Also, the processing module 170 may have one or more input and output devices, including the touch display 180, which can be used, among other things, to present a user interface. Examples of output devices that can be used to provide a user interface include the touch display 180 as well as printers or display screens for visual presentation of output and speakers or other sound generating devices for audible presentation of output. Examples of input devices that can be used for a user interface include the touch display 180, keyboards, and pointing devices, such as mice, touch pads, and digitizing tablets. As another example, a computer may receive input information through speech recognition or in other audible format.

The processing module 170 may be connected to one or more computer or information-sharing networks, including a local area network or a wide area network, such as an enterprise network, an intelligent network (IN), or the Internet. Such networks may be based on any suitable technology and may operate according to any suitable protocol and may include wireless networks, wired networks, or fiber optic networks.

OLED Arrays for Illuminating Cells in Glassware and Plasticware

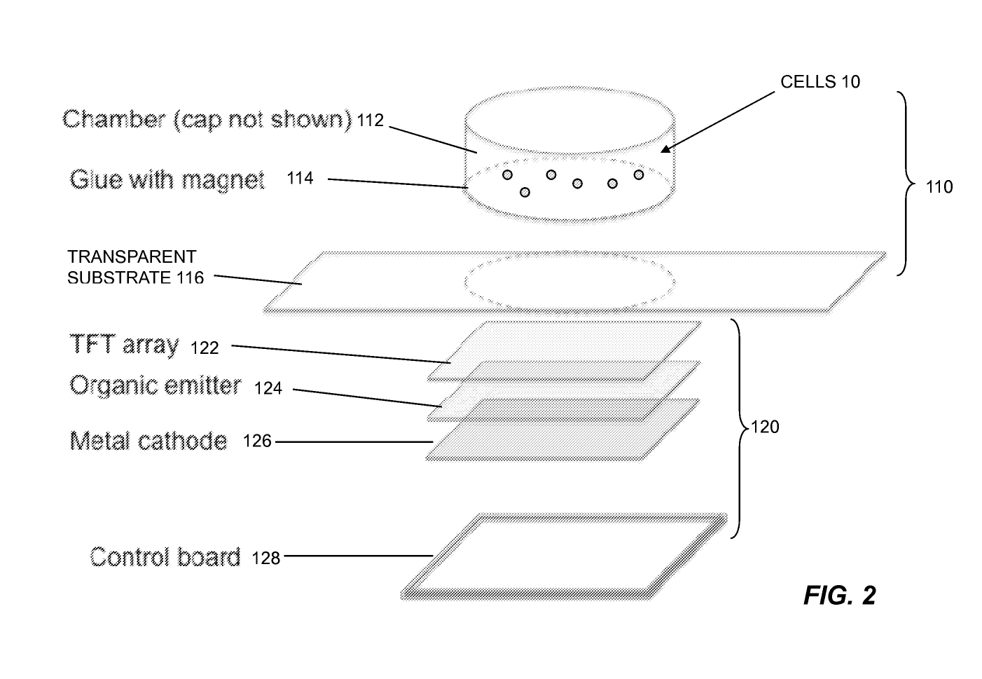

FIG. 2 illustrates the cell culture dish 110 and the OLED array 120 of FIG. 1 in greater detail. The cell culture dish 110 and the OLED array 120 can be manufactured as separate components or a single integrated component. As will be readily appreciated by those of skill in the art, they can be used in a wide variety of applications, including conventional microscopy, lensless imaging, cell characterization, cell manipulation, cell separation/sorting, and the cell culturing and tracking system 100 shown in FIG. 1.

The cell culture dish 110 may be formed of any suitable material, including glassware and plasticware, that is at least partially transparent at the wavelength(s) of light emitted by the OLED array 120. It can be fabricated from a single piece of material or, as shown in FIG. 2, from two or more pieces of material, such as a chamber 112 held on a transparent substrate 16 with a magnet 116 and/or adhesive (for example, glue or ultraviolet-cured epoxy) and topped with an optional cap (not shown). Depending on the application, the transparent substrate 16 may be rigid or flexible, and can be made of glass, plastic, polymer film, or any other suitable material(s). (Note that the magnet 116 in glue is a demonstration for the joining of a separate AMOLED unit with the glassware or plasticware.) One or more of the cell culture dish's interior surfaces may be textured, patterned, treated, or otherwise modified to promote or enhance adhesion of cells 10 and/or cell culture growth. The cell culture dish 110 may be any suitable shape or size, and may take the form of a chamber slide, microplate (for example, for high throughput screening), Petri dish, or plate. Cell culture dish 110 can generally be any shape. Common shapes include circles, squares, rectangles, hexagons, and other geometrical shapes.

The OLED display 120 comprises an organic emitter 124 sandwiched between a metal cathode 126 and a thin-film transistor (TFT) array 122, which forms part of an active matrix addressing system. In some examples, each of these layers may be relatively thin, and the OLED's total thickness may be about 200 nm to about 300 nm. A control board 128 forms another part of the active matrix addressing system. The organic emitter 124 may include a single type of material that emits light at particular wavelength (for example, blue light) or over a particular range of wavelengths. It may also include several different types of materials, possibly arranged in a striated or pixelated pattern, each of which emits light at a different wavelength (for example, red, green, blue) or over a particular range of wavelengths when stimulated with electric current. For instance, each pixel may be divided into a plurality of sub-pixels, and the chemical structure of the organic emitter material in each sub-pixel may be altered to emit red, green, or blue light. The pixel's brightness is adjusted by altering the current to each sub-pixel, with the ratios of current in the red green and blue sub-pixels determining the overall color of the pixel. If desired, the organic emitter 124 may be distributed over a regular area (for example, a polygonal area) or an amorphous area. In addition, the organic emitter 124 may be distributed on a planar surface, a faceted surface, and/or a warped/curve surface.

As readily understood by those of skill in the art, the OLED array 120 may be subdivided in multiple regions, commonly called picture elements or “pixels.” The size(s), number, and arrangement of pixels in the OLED array 120 can be chosen based on the application and/or the desired resolution. For instance, one or more pixels in the OLED array 120 may be about the size of a eukaryotic cell. The OLED array 120 may have hundreds, thousands, or millions of pixels. For instance, the OLED array 120 may include about 5.4 million pixels and extend over a rectangular area with a 0.67-inch (17 mm) diagonal. The OLED array's subpixel pitch may be 4.7 μm×4.7 μm, which corresponds to a pixel size that, at less than 4.7 μm, is smaller than the size of most somatic cells in human body (for example, about 10 μm to about 150 μm). (The smallest somatic cell is an anuclei red blood cell at 5 μm, whereas the smallest human cell is the sperm cell at 3 μm.) Because the pixels may be so small, they can be used to improve the resolution for tracking individual cells, for example, to a fraction of a cell size.

As understood by those of skill in the art, each pixel in the OLED array 120 is controlled by corresponding unit cell in the TFT array 122. Generally speaking, each unit cell in the TFT array 122 may include one or more TFTs configured to control the current and/or voltage applied to the organic emitter 124 in the pixel. A TFT is a field-effect transistor made by depositing thin films of a semiconductor active layer as well as a dielectric layer and metallic contacts over a supporting substrate. In some examples, the substrate is glass or transparent and flexible plastic. This differs from a conventional transistor where the semiconductor material typically is the substrate, such as a silicon wafer. The TFT array 122 is coupled to the control board 128, which enables the user to switch each pixel on and off independently, either by manipulating an input/output interface (for example, touch display 180 in FIG. 1) or view pre-programmed instructions executed by a processor (for example, processor 170 in FIG. 1). The control board 128 enables the user to control the wavelength, intensity, and duration of illumination provided by each pixel in the OLED display 120. For instance, FIG. 3 shows an OLED display 120 that is actuated via the control board 128 to emit light in a predetermined pattern—in this case, to form the letter “g” and “u.”

If desired, the OLED array 120 may be directly printed on a surface of the transparent substrate 116 opposite the chamber 112. For instance, the OLED array 120 may be printed or deposited onto the transparent substrate 116 using screen-printing, lithography (for example, photolithography), or any other suitable technique. Printing technology is particularly attractive because it is readily available, reasonably inexpensive, and can be used to make large OLED displays relatively quickly (for example, a 50-inch (127 cm) display in less than 2 minutes). Alternatively, the OLED array 120 may be an independent component, in which case it may be joined with or to a cell culture apparatus using embedded magnets, adhesive, clips, clamps, fasteners (for example, screws), and/or any other suitable fixture device.

Forming the OLED array 120 on the transparent substrate 116 eliminates air between the transparent substrate 116 and the OLED array 120. Because there is no gap between the transparent substrate 116 and the OLED array 120, the device can be more compact, and may even fit inside an incubator (for example, as shown in FIG. 1). The resulting proximity of the OLED array to the cell culture dish 110 (and the cells 10 themselves) may also improve the resolution of the optical system without the need for additional optical components.

### EXAMPLES

The following examples are provided to illustrate aspects of the present disclosure. The examples are not intended to limit the scope of the claims.

### Example 1

### Stem Cell Differentiation and Migration

In one example, a cell culturing and tracking system can be used to study the differentiation and migration of stem cells in the presence of one or more factors for early cell fate decisions, such as transcription factors and mitogens capable of inducing and/or directing stem cell differentiation and migration. For instance, it can be used to observe the migration of CXCR4-expressing mesenchymal stem cells in the presence of SDF-1. While data show that stem cells are attracted to the mitogen as a result of chemotaxis, the distance of migration for most cells is within about 10 cell radii from the origin of seeding. This finding implies that it would be difficult to measure and quantify the cell movement in real time because the resolution of cell migration is less than 1 mm, which may be too small to be visualized between the marks at the bottom of each cell culture slide or plate using conventional techniques.

In contrast, a cell culturing and tracking system like the one shown in FIG. 1 can track the cells within an array of OLEDs printed on the culturing dish opposite the cell culture chamber. The OLEDs in the OLED array have a pixel pitch of about 5 μm, which is on the order of the average stem cell radius and about an order of magnitude smaller than the stem cell migration distance. Light from the OLED array illuminates the stem cells, and a detector array opposite the stem cells from the OLED array detects the transmitted beam. Like the OLED array, the detector array has a pixel pitch equal to or smaller than the stem cell and migration distance, enabling detection of migration on a cell-by-cell basis.

### Example 2

### Screening Assay for Ligand Binding

In some embodiments, the present technology is useful for studying ligand binding to cell surface receptors. For example, the present technology can be used to study the interaction of a test ligand with a target receptor. The target receptors on the cell surfaces are labeled with fluorescent markers, then the cells are cultured in a cell culture dish with an OLED attached to the bottom of the dish. The cells are incubated with a fluorescent labeled test ligand that emits green light and the target receptor's fluorescent label emits red light.

The emission of the proper wavelength of light from the OLED will cause fluorescence of the test ligand and the target receptor. The presence of red fluorescence on the cell surface indicates the presence of the target receptor. The presence of green fluorescence indicates the presence of the test ligand. Comparison of the location of the fluorescence indicates whether the test ligand is bound to the target receptor. If the green fluorescence is not in contact with the red fluorescence, then the ligand did not bind to the target receptor. The presence of green fluorescence in contact with the red fluorescence indicates that the ligand is bound to the receptor.

Additionally, whether the test ligand is an agonist or antagonist can also be determined. If the test ligand is an agonist, the binding of the test ligand to the target receptor causes the target receptor to translocate to the nucleus. If the test ligand is an antagonist, the binding of the test ligand to the target receptor causes the target receptor to stay on the cell surface. Thus, if light emitted from the OLED shows red fluorescence in the nucleus, then the test ligand activates the target receptor. Conversely, if the red fluorescence remains on the cell surface, then the test ligand prevents activation of the target receptor.

The present technology is efficient for screening assays for ligand binding as real-time images can be produced in series to follow the ligand binding process from the initial binding to the receptor to the internalization of the receptor, to the nuclear translocation of the receptor, without staining and fixing cells.

### Example 3

### Assay for Enzymatic Activity

In another example, an OLED-based cell culturing apparatus is used for studying enzymatic activity. A fluorescent labeled substrate for a target enzyme is delivered into cells. Fluorescence is only detected if the substrate is cleaved. The cells are incubated with the test compound. If the test compound inhibits the target enzyme, then substrate cleavage is low and little, if any, fluorescence is detected when the OLED emits light under a cell or group of cells. If the test compound does not inhibit the enzyme, then substrate cleavage occurs and fluorescence is detected when the OLED emits light under a cell or group of cells. If the test compound enhances enzyme activity, then a large amount of substrate are cleaved and a high amount of fluorescence is detected by emission of light by the OLED under a cell or group of cells.

The OLED-based cell culturing apparatus can also be used to capture a series of real-time images that provide data on the effects of an inhibitor. For example, it can be used track changes in the fluorescence intensity of a single cell or group of cells over time. This data can be used to determine the time taken to decrease fluorescence after treating cells containing a cleavable fluorescent substrate with an inhibitor.

### Example 4

### Cell Proliferation

An OLED-based cell culture apparatus can also be used to study the effect of a test compound on cell proliferation. A target cell is labeled with a fluorescent marker, then cultured in a cell culture dish having an OLED attached to the bottom of the dish. The target cells are incubated with the test compound and monitored with OLED illumination to determine if the test compound increased cell proliferation as indicated by detection of more fluorescent cells in real-time. The data can also be used to determine if the test compound prevents cell proliferation, as indicated by a steady-state fluorescence signal, or promoted cell apoptosis, as indicated by a severe decrease in the number of detectable fluorescent cells. The ability to track a single or small group of cells reduces the need for a large number of cells to be tested and provides a method for efficient high-throughput screening of compounds.

### Example 5

### High-Throughput Screening of siRNA

Another example of the OLED-based cell culturing and screening apparatus is used in high-throughput siRNA screening. Cells are cultured in a 96-well microtiter plate. Next, at least one fluorescent labeled siRNA is transfected into the cells each well. A first set of wells is transfected with the same siRNA or same group of siRNAs and second set of wells is transfected with a different siRNA or different group of siRNAs. After transfection, the effect of the siRNA or group of siRNAs is analyzed by imaging individual cells or groups of cells. Fluorescent detection by OLED may be used to identify which cells were transfected with the siRNA and the amount of siRNA transfected. Unlike other solution, the OLED-based cell culturing and screening apparatus promotes efficiency in high-throughput screening, as adherent cells do not have to be trypsinized or subjected to flow cytometry, both of which can lower yield of viable cells, to determine which cells were successfully transfected.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (for example, a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, and so on that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

The use of flow diagrams is not meant to be limiting with respect to the order of operations performed. The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations.

However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. A system for illuminating at least one cell, the system comprising:
a transparent substrate having a thickness of between about 10 nm to about 100 μm, wherein the transparent substrate defines a first surface to support the at least one cell and a second surface opposite the first surface from the at least one cell;
an array of organic light-emitting diodes (OLEDs), in optical communication with the at least one cell via the transparent substrate, configured to illuminate the at least one cell with light, wherein the array of OLEDs is substantially adjacent the second surface of the transparent substrate; and
a controller, operably coupled to the array of OLEDs, configured to control at least one of an intensity and a wavelength of the light emitted by the array of OLEDs.

2. (canceled)

3. (canceled)

4. The system of claim 1, wherein the array of OLEDs is in contact with the second surface of the transparent substrate.

5. The system of claim 1, wherein the array of OLEDs is printed on the second surface of the transparent substrate.

6. The system of claim 1, wherein the first surface at least partially defines a cavity configured to hold the at least one cell.

7. The system of claim 1, wherein the first surface is configured to support adhesion of the at least one cell to the first surface.

8. The system of claim 1, wherein the second surface comprises a curved portion and wherein the array of OLEDs extends at least partially over the curved portion.

9. The system of claim 1, wherein the array of OLEDs has a pitch of between about 10 nm to about 50 μm.

10. The system of claim 9, wherein the controller comprises:
a thin-film transistor layer, operably coupled to the array of OLEDs, configured to actuate at least one OLED in the array of OLEDs.

11. The system of claim 10, wherein the array of OLEDs comprises:
at least one first OLED configured to illuminate the at least one cell at a first wavelength; and
at least one second OLED configured to illuminate the at least one cell at a second wavelength.

12. The system of claim 1, further comprising:
a detector, in optical communication with the particle, configured to provide a signal representative of radiation transmitted through or emitted by the at least one cell; and
a processor, operably coupled to the detector, configured to identify a parameter of the at least one cell based at least in part on the signal.

13. The system of claim 12, wherein the parameter includes at least one of a size, a position, a fluorescence wavelength, a fluorescence intensity, a speed, and a trajectory of the at least one cell.

14. A method of illuminating at least one cell, the method comprising:
providing at least one cell in optical communication with a transparent substrate, wherein the transparent substrate defines a first surface to support the at least one cell and a second surface opposite the first surface from the at least one cell;
illuminating the at least one cell with light transmitted through the transparent substrate from an array of organic light-emitting diodes (OLEDs), wherein the array of OLEDs is in contact with the second surface of the transparent substrate; and
modulating at least one of an intensity and a wavelength of the light transmitted through the transparent substrate from the array of OLEDs.

15. The method of claim 14, wherein providing the at least one cell comprises at least one of:
disposing the at least one cell in a cavity at least partially defined by the transparent substrate;
flowing the at least one cell over a surface of the transparent substrate; and
allowing the at least one cell to adhere to the surface of the transparent substrate.

16. The method of claim 14, wherein the array of OLEDs comprises a first OLED and a second OLED spaced at a pitch of about 10 nm to about 50 μm, and wherein illuminating the cell culture comprises:
emitting a first portion of the light from the first OLED; and
emitting a second portion of the light from the second OLED.

17. The method of claim 16, wherein illuminating the at least one cell further comprises:
actuating the first OLED with a transistor in a thin-film transistor layer operably coupled to the array of OLEDs.

18. The method of claim 16, wherein illuminating the at least one cell further comprises:
emitting the first portion of the light at a first wavelength from the first OLED;
emitting the second portion of the light at a second wavelength from the second OLED.

19. The method of claim 18, further comprising:
detecting radiation transmitted through or emitted by the at least one cell;
providing an electromagnetic signal representative of the radiation; and
identifying a parameter associated with the at least one cell based at least in part on the electromagnetic signal, and wherein identifying the parameter associated with the at least one cell comprises estimating at least one of a size, a position, a speed, a fluorescence wavelength, a fluorescence intensity, and a trajectory of the at least one cell.

20. (canceled)

21. A system for culturing and/or tracking at least one cell, the system comprising:
a transparent substrate having a first surface configured to at least partially support the at least one cell and a second surface opposite the first surface;
a two-dimensional array of organic light-emitting diodes (OLEDs), disposed on the second surface of the transparent substrate, configured to illuminate the at least one cell;
an active matrix layer, in electrical communication with the two-dimensional array of OLEDs, configured to actuate at least one OLED in the two-dimensional array of OLEDs;
a detector, in optical communication with the at least one cell, configured to sense light transmitted, reflected, scattered, and/or emitted by the at least one cell; and
a processor, operably coupled to the active matrix layer and the detector, configured to control the two-dimensional array of OLEDs based at least in part on the light sensed by the detector.

22. The system of claim 21, wherein the two-dimensional array of OLEDs comprises a plurality of OLEDs at a pitch of about 1 μm to about 50 μm.

23. The system of claim 21, wherein the two-dimensional array of OLEDs comprises at least one first OLED configured emit light at a first wavelength and at least one second OLEDs configured to emit light at a second wavelength.

24. The system of claim 21, wherein the active-matrix layer comprises a plurality of a thin-film transistors configured to actuate the two-dimensional array of OLEDs.
